(19) United States
(12) Patent Application Publication
(10) Pub. No.: US 2024/0233909 A1
(43) Pub. Date: Jul. 11, 2024

(54) SYSTEM FOR PROVIDING FOOD

(71) Applicant: Naga Lalitha Kumar Mudiganti, Cary, NC (US)

(72) Inventor: Naga Lalitha Kumar Mudiganti, Cary, NC (US)

(21) Appl. No.: 18/612,013

(22) Filed: Mar. 21, 2024

Related U.S. Application Data
(63) Continuation-in-part of application No. 17/667,121, filed on Feb. 8, 2022, now abandoned.

(51) Int. Cl. G16H 20/60

(52) U.S. Cl. CPC G16H 20/60

(57) ABSTRACT

A system for providing food to consumers using a centralized system wherein members of the system employ a complete chain of cooking raw food, consuming the cooked food, delivering the cooked food, and procuring supplies, wherein there is at least one of the group of people that does each of the steps together or independently, which subsequently reduces the costs and efforts associated in delivering cooked meals.

This application is a continuation-in-part of U.S. non-provisional patent application Ser. No. 17/667,121 filed on Feb. 8, 2022, which is incorporated herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and

Trademark Office patent files or records, but otherwise reserves all copyright rights whatsoever.

### BACKGROUND OF THE INVENTION

### Field of the Invention

The present invention relates to a system for providing food to consumers, as well as nutritional information, in a centralized system, wherein members of the system work together as one system.

### Description of Related Art

The providing of meals to individual homes has become unwieldy and very expensive. Food delivery services allow users to have their food delivered, but not only is there a delivery charge, the cost of the food purchased is also raised. In addition, if different cuisines are desired, one has to make multiple orders from multiple different restaurants.

The isolation of a restaurant serving one cuisine adds to the cost even further. Ordering and acquiring produce, employing staff/workers, along with the cost of delivery creates higher fees and is subsequently more expensive. For example, if there are 10 restaurants, there will be 10 different costs/charges which will only increase depending on proximity (i.e., how far they are from the source and the customers). In addition, restaurants don't provide precise food data like nutritional information, especially if food is ordered from multiple locations.

### BRIEF SUMMARY OF THE INVENTION

The present invention relates to a system for providing cooked food within an organized group of people in a manner that reduces cost and effort to deliver cooked meals as well as provide precise food data for orders from multiple locations.

Accordingly, in one embodiment, there is a system for providing food to a consumer comprising:
a) an online ordering system that can take prepared food orders from a consumer wherein the prepared food is ordered from a plurality of locations and wherein there is an organized group of people that cook raw foods into the prepared food at each of the plurality of locations;
b) tracking all the individual ingredients in the prepared food that is ordered; and
c) providing precise food data of all the individual ingredients in the prepared foods ordered by the customer.

### BRIEF DESCRIPTION OF THE DRAWINGS

FIG. 1 is a relationship chart of the various elements of the present invention.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### DEFINITIONS

The terms “about” and “essentially” mean ±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “system for providing food to a consumer” refers to a system for providing cooked meals to customers with nutritional content wherein the system includes the provisions of raw food, people who cook the raw food, consume the cooked food, deliver the cooked food and procure supplies for cooking the food, wherein, the people are all members of an organized group where at least one of the group of people is responsible for one or more of the elements of the system. By having a closed system, food can be provided at a lower cost than traditional ways of providing and delivering food, such as Grubhub®, which delivers a single meal from a selection of hundreds of restaurants not connected to the consumer.

As used herein, the term “geographically connected organized group of people” refers to the people in the organized group that are part of the system and are all geographically close to one another, such as in a city neighborhood. And that the group of people in the system all fit this requirement.

As used herein, the term “raw food” refers to one or more ingredients that needs to be cooked or otherwise prepared before serving.

As used herein, the term “cook the raw food” refers to one or more of the people of the organized group being responsible for cooking and preparing the meals of the system. This can be done in a professional kitchen or in one embodiment, from a member(s) kitchen or a set up kitchen in any location. All of the ingredients do not to be raw, but a plurality of ingredients is contemplated in one embodiment.

As used herein, the term “consume the cooked food” refers to one or more of the people of the organized group who eat the food that is being prepared.

As used herein, the term “deliver the cooked food” refers to one or more of the people of the organized group driving the cooked food in personal or other vehicles to the consuming customer. In one embodiment, instead of one restaurant with one delivery per vehicle, routes can be established allowing the delivery to be for multiple locations for meal drop off.

As used herein, the term “procure supplies” refers to one or more of the people of the organized group finding all the necessary equipment accessories and the like for the cooking, storing, and delivering of the cooked food.

As used herein, the term “group of people that does each of the steps together or independently” refers to one or more of the people of the organized group optionally doing more than one function. For example, one individual can be responsible for both cooking the food and delivering the food.

As used herein, the term “online ordering system” refers to people who consume the food going online to a website or the like to order food from different locations for delivery and food data.

As used herein, the term “plurality of locations” refers to having multiple places where the cooking is done. In one embodiment, it is in peoples' home kitchens. One could have different locations for each type of food or just split up/divide the work. Other locations could be a rented kitchen, a temporary kitchen, and the like.

As used herein, the term “plurality of people” refers to having multiple people from the group to cook the raw food, consume the food, deliver the food, and procure supplies. While that could all be one person, in one embodiment it is 5, 10, 15, or 20 people.

As used herein, the term “home kitchens” refers to cooking the raw food in one or more home kitchens belonging to the group of people. One advantage of the home kitchen is the much-reduced cost of food preparation. In addition, the person cooking the food will already be familiar with the kitchen, thus preventing a learning curve that occurs when using a new kitchen.

As used herein, the term “route that delivers to multiple locations” refers to delivery accomplished by grouping several meals for distribution to more than one location, as opposed to the one driver-one dish approach used by current delivery services

As used herein, the term “additional connections” refers to finding other pieces of the utilization of the system such as where to buy food, equipment, attain new customers, and the like.

As used herein the term “tracking all the individual ingredients” refers to compiling a list of the quantity each ingredient for all the items ordered from multiple locations. This includes additions like condiments, more or less ingredients and the like.

As used herein the term “food data” refers to information on the food as delivered including nutritional content, taste, environmental impact of the food, the food experience, and the like.

As used herein the term “taste” refers to the overall experience in eating the food. This includes information on sweet, salt, umami, aroma, temperature, and the like.

As used herein the term “environmental impact” refers to the effects of the preparation of the food including pollution, economic cost, other things which affect the environment as well as the community effects and health effects.

As used herein “the food experience” refers to experience during the consumption of food including aroma, texture, taste, and the like.

As used herein the term “providing nutritional content” refers to gathering all the nutritional information for all the raw food in the customer order. This information includes at least one of total weight, cost, total fat, total cholesterol, total sodium, total dietary fiber, total sugars, total protein, and total calories. Since use of raw foods is contemplated and will vary from preparation to preparation, all of the food used must be precisely weighed to calculate the precise nutritional information.

### DRAWINGS

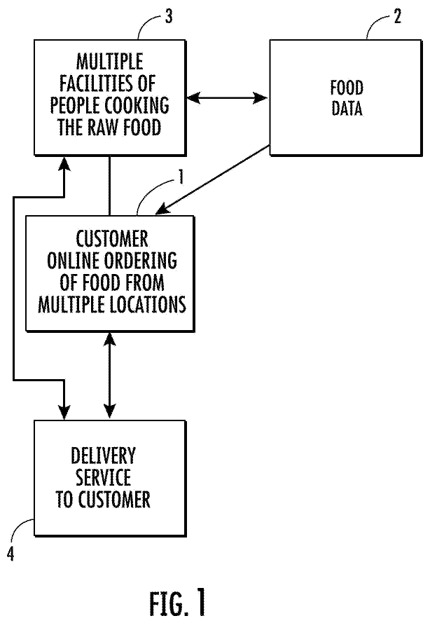

Now referring to the drawings, FIG. 1 is a graphic representation of the system of the present invention. In this view, the raw food is delivered to the multiple locations 3 where people cook the raw food. Once the raw food is cooked 3, it is taken by the delivery service 4 for delivery to the customer 1. Ordering and coordination of the system is provided by online ordering system 1. Food data 2 is delivered to the customer 1 based on all the ingredients in the customer's order.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A system for providing food to a consumer comprising:
a) an online ordering system that can take prepared food orders from a consumer wherein the prepared food is ordered from a plurality of locations and wherein there is an organized group of people that cook raw foods into the prepared food at each of the plurality of locations;
b) tracking all the individual ingredients in the prepared food that is ordered; and
c) providing precise food data of all the individual ingredients in the prepared foods ordered by the customer.

2. The system for providing food to a consumer according to claim 1, wherein the plurality of locations includes home kitchens.

3. The system for providing food to a customer according to claim 1 wherein the food data is the nutritional content.

4. The system for providing food to a consumer according to claim 3 wherein the nutritional content includes at least one of total fat, total cholesterol, total sodium, total dietary fiber, total sugars, total protein and total calories.

5. The system for providing food to a customer according to claim 1 wherein the food data is at least one of the data comprising taste, nutrition, environmental impact of the food, and the experience of the food.
